(19) United States
(12) Patent Application Publication
(10) Pub. No.: US 2025/0111933 A1
(43) Pub. Date: Apr. 3, 2025

(54) EXAMINATION SUPPORT DEVICE, OPERATION METHOD OF EXAMINATION SUPPORT DEVICE, PROGRAM, AND MEDICAL IMAGE CAPTURING APPARATUS

(71) Applicant: FUJIFILM Corporation, Tokyo (JP)

(72) Inventor: Takashi AOSHIMA, Chiba (JP)

(73) Assignee: FUJIFILM Corporation, Tokyo (JP)

(21) Appl. No.: 18/898,627

(22) Filed: Sep. 26, 2024

(30) Foreign Application Priority Data
Sep. 29, 2023 (JP) 2023-170721

(51) Int. Cl. G16H 30/40; A61B 5/055

(52) U.S. Cl. CPC G16H 30/40; A61B 5/055

(57) ABSTRACT

Provided are an examination support device, an operation method of an examination support device, a program, and a medical image capturing apparatus with which a load on an operator in creation of an examination protocol is reduced. An examination support device acquires an examination order for a subject of a medical image diagnostic examination, acquires subject information regarding the subject, acquires examination information regarding the medical image diagnostic examination performed on the subject, and creates a first examination protocol applied to the medical image diagnostic examination performed based on the examination order, based on the subject information and the examination information.

### CROSS-REFERENCE TO RELATED APPLICATION

The present application claims priority under 35 U.S.C § 119 (a) to Japanese Patent Application No. 2023-170721 filed on Sep. 29, 2023, which is hereby expressly incorporated by reference, in its entirety, into the present application.

### BACKGROUND OF THE INVENTION

### 1. Field of the Invention

The present disclosure relates to an examination support device, an operation method of an examination support device, a program, and a medical image capturing apparatus.

### 2. Description of the Related Art

In a case in which imaging is performed by using a medical image capturing apparatus, an examination order created in a hospital information system is transmitted to an image diagnosis department information management system. In the image diagnosis department information management system, various types of information regarding an examination are input to the examination order and transmitted to the medical image capturing apparatus. In the medical image capturing apparatus, an examination protocol is set based on the examination order, and imaging of a subject is performed based on the set examination protocol.

It should be noted that the medical image capturing apparatus can be referred to as a modality. The hospital information system can be referred to as HIS using an abbreviation for hospital information system that is the English notation. The image diagnosis department information management system can be referred to as RIS using an abbreviation for radiology information system that is the English notation.

JP2020-187542A describes an imaging support device having a function of automatically determining an imaging protocol based on an examination order or the like and transmitting the determined imaging protocol to a modality. In the device described in JP2020-187542A, a trained learning model is used in a case of determining the imaging protocol. The learning model used in a case of determining the imaging protocol is a trained learning model that has been trained using a change element set including patient information, an examination order, and an examination instruction as training data and using an imaging protocol used in the training data as correct answer data.

In the operation of the learning model, a change factor set extracted from each of the patient information, the examination order, and the examination instruction is input from the image diagnosis department information management system to the learning model, and the imaging protocol is output from the learning model. In the modality, the imaging protocol output from the learning model is applied, and the imaging is performed.

In addition, in the device described in JP2020-187542A, in a case in which the imaging protocol transmitted from the image diagnosis department information management system is changed in the modality and the two imaging protocols are not matched, a correspondence relationship between the imaging protocol and the contents, such as the patient information, the examination order, and the examination instruction, is updated.

### SUMMARY OF THE INVENTION

However, in the medical image capturing apparatus, in a case in which the examination protocol transmitted from the image diagnosis department information management system is changed, an operator, such as a radiologist, is required to perform the confirmation of the examination order, the creation of the examination protocol, the confirmation of the examination protocol, and the like, and thus there is a need to reduce a burden on the operator.

JP2020-187542A describes that the imaging protocol transmitted from the image diagnosis department information management system to the modality is changed in the modality, but does not specifically describe how the imaging protocol is changed in the modality.

The present disclosure has been made in view of such circumstances, and an object of the present disclosure is to provide an examination support device, an operation method of an examination support device, a program, and a medical image capturing apparatus with which the load on the operator in the creation of the examination protocol is reduced.

A first aspect of the present disclosure relates to an examination support device comprising: one or more processors; and one or more memories that store a program including one or more commands executed by the one or more processors, in which the one or more processors execute the program stored in the one or more memories to: acquire an examination order for a subject of a medical image diagnostic examination; acquire subject information regarding the subject; acquire examination information regarding the medical image diagnostic examination performed on the subject; and create a first examination protocol applied to the medical image diagnostic examination performed based on the examination order, based on the subject information and the examination information.

With the examination support device according to the first aspect, the first examination protocol is generated based on the subject information regarding the subject of the examination and the examination information regarding the medical image diagnostic examination. As a result, the load on the operator in the creation of the examination protocol is reduced.

The first examination protocol can include a first imaging protocol applied to capturing of a medical image of the subject of the examination. In addition, the first examination protocol can include a sequence related to processing on the medical image.

A second aspect relates to the examination support device according to the first aspect, in which the one or more processors may acquire a second examination protocol associated with the examination order, and create the first examination protocol by modifying the second examination protocol based on the examination information and the subject information.

According to such an aspect, the first examination protocol in which a defect of the examination order or the like is modified can be generated.

A third aspect relates to the examination support device according to the second aspect, in which the one or more processors may display, on a display device, a comparison result obtained by comparing the first examination protocol with the second examination protocol.

According to such an aspect, the operator or the like can visually recognize a difference between the first examination protocol and the second examination protocol.

A fourth aspect relates to the examination support device according to the second or third aspect, in which the one or more processors may perform at least any one of substitution of a sequence, order change of a sequence, addition of a sequence, or deletion of a sequence in the second examination protocol in a case of creating the first examination protocol.

According to such an aspect, the modification can be performed in units of a sequence constituting the examination protocol.

A fifth aspect relates to the examination support device according to any one of the first to fourth aspects, in which the one or more processors may acquire past examination information regarding a past medical image diagnostic examination performed in past on the subject, and create the first examination protocol based on the subject information, the examination information, and the past examination information.

According to such an aspect, the first examination protocol in which the past examination information is reflected can be created.

A sixth aspect relates to the examination support device according to any one of the first to fifth aspects, in which the one or more processors may display, on a display device, an authentication screen for an operator to input whether or not to approve the first examination protocol.

According to such an aspect, the operator who visually recognizes the authentication screen can input the approval or the disapproval of the first examination protocol.

A seventh aspect relates to the examination support device according to any one of the first to sixth aspects, in which the one or more processors may receive modification of the created first examination protocol.

According to such an aspect, the defect of the first examination protocol can be modified.

An eighth aspect relates to the examination support device according to the seventh aspect, in which the one or more processors may store a modification history of the first examination protocol.

According to such an aspect, the modification history of the first examination protocol can be used.

A ninth aspect of the present disclosure relates to an operation method of an examination support device, the operation method comprising: acquiring an examination order for a subject of a medical image diagnostic examination; acquiring subject information regarding the subject; acquiring examination information regarding the medical image diagnostic examination performed on the subject; and creating a first examination protocol applied to the medical image diagnostic examination performed based on the examination order, based on the subject information and the examination information.

With the operation method of an examination support device according to the ninth aspect of the present disclosure, the same actions and effects as those of the examination support device according to the first aspect can be obtained. Configuration requirements of the examination support device according to the second to eighth aspects can be applied as configuration requirements of the operation method of an examination support device according to the other aspect.

A tenth aspect of the present disclosure relates to a program causing a computer to realize: a function of acquiring an examination order for a subject of a medical image diagnostic examination; a function of acquiring subject information regarding the subject; a function of acquiring examination information regarding the medical image diagnostic examination performed on the subject; and a function of creating a first examination protocol applied to the medical image diagnostic examination performed based on the examination order, based on the subject information and the examination information.

With the program according to the tenth aspect of the present disclosure, the same actions and effects as those of the examination support device according to the first aspect can be obtained. Configuration requirements of the examination support device according to the second to eighth aspects can be applied as configuration requirements of the program according to the other aspect.

An eleventh aspect of the present disclosure relates to a medical image capturing apparatus comprising: an examination support device that supports capturing of a medical image, in which the examination support device includes: one or more processors; and one or more memories that store a program including one or more commands executed by the one or more processors, the one or more processors execute the program stored in the one or more memories to: acquire an examination order for a subject of a medical image diagnostic examination; acquire subject information regarding the subject; acquire examination information regarding the medical image diagnostic examination performed on the subject; and create a first examination protocol applied to the medical image diagnostic examination performed based on the examination order, based on the subject information and the examination information.

With the medical image capturing apparatus according to the eleventh aspect of the present disclosure, the same actions and effects as those of the examination support device according to the first aspect can be obtained. Configuration requirements of the examination support device according to the second to eighth aspects can be applied as configuration requirements of the medical image capturing apparatus according to the other aspect.

According to the present disclosure, the first examination protocol is generated based on the subject information regarding the subject of the examination and the examination information regarding the medical image diagnostic examination. As a result, the load on the operator in the creation of the examination protocol is reduced.

### BRIEF DESCRIPTION OF THE DRAWINGS

FIG. 1 is a block diagram showing a configuration example of a medical information system.

FIG. 2 is an overall configuration diagram of an MRI apparatus applied to the medical information system shown in FIG. 1.

FIG. 3 is a functional block diagram showing an electric configuration of an examination support device according to a first embodiment.

FIG. 4 is a block diagram showing a hardware configuration of the electric configuration of the examination support device shown in FIG. 3.

FIG. 5 is a flowchart showing a procedure of an examination support method according to the first embodiment.

FIG. 6 is a table showing a correspondence relationship between examination information and a change content of an examination protocol.

FIG. 7 is a table showing a correspondence relationship between subject information and the change content of the examination protocol.

FIG. 8 is a schematic diagram showing a configuration example of a user interface.

FIG. 9 is a functional block diagram showing an electric configuration of an examination support device according to a second embodiment.

FIG. 10 is a flowchart showing a procedure of an examination support method according to the second embodiment.

### DESCRIPTION OF THE PREFERRED EMBODIMENTS

Hereinafter, preferred embodiments of the present invention will be described in detail with reference to the accompanying drawings. It should be noted that, in the following description and the accompanying drawings, the same reference numerals denote the same constituent elements, and the duplicated description thereof is omitted. In the following embodiments, in a case in which a plurality of constituent elements are described and listed, it can be interpreted that at least one of the plurality of constituent elements is included.

### Configuration Example of Medical Information System

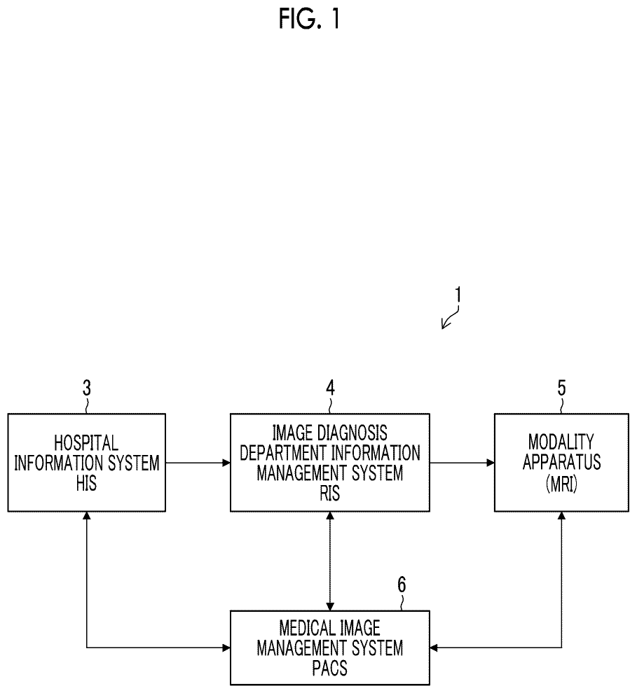

FIG. 1 is a block diagram showing a configuration example of a medical information system. A medical information system 1 shown in FIG. 1 is an information processing system that is introduced into a medical facility such as a hospital. The medical information system 1 comprises a hospital information system 3, an image diagnosis department information management system 4, a modality apparatus 5, and a medical image management system 6. It should be noted that the medical image management system can be referred to as PACS using an abbreviation for picture archiving and communication systems.

The hospital information system 3 is a system that manages various types of business in the entire medical facility. The hospital information system 3 can include a plurality of systems, such as an electronic medical record system, an ordering system, and a medical accounting system.

The image diagnosis department information management system 4 is a system that manages the business of an image diagnosis department, such as a radiology department. The image diagnosis department information management system 4 is linked with the hospital information system 3 to perform processing such as examination reservation of the image diagnostic examination, creation of an examination order, and management of an examination result.

The modality apparatus 5 is a medical image capturing apparatus that captures a medical image of a subject. The modality apparatus 5 captures the medical image based on the examination order transmitted from the hospital information system 3. The examination order transmitted from the hospital information system 3 to the modality apparatus 5 is associated with an examination protocol. The modality apparatus 5 performs the capturing of the medical image in accordance with an examination protocol associated with the examination order.

FIG. 1 shows an MRI apparatus as the modality apparatus 5. Examples of the modality apparatus 5 include a CT apparatus, an ultrasound diagnostic apparatus, a PET apparatus, a mammography apparatus, an X-ray diagnostic apparatus, an X-ray fluoroscopic diagnostic apparatus, and an endoscope apparatus.

It should be noted that MRI is an abbreviation for magnetic resonance imaging that is the English notation. CT is an abbreviation for computed tomography. PET is an abbreviation for positron emission tomography. The subject according to the embodiment is an example of a subject of a medical image diagnostic examination.

The medical information system 1 may be provided with a plurality of modality apparatuses 5. The plurality of modality apparatuses 5 may be of the same type as or different types from each other. The number and the type of the modality apparatuses 5 are appropriately determined for each medical facility.

The medical image management system 6 is a computer system that stores examination data captured by using the modality apparatus 5 and including the generated medical image and that manages the examination data. The medical image management system 6 comprises a large-capacity external storage device and a program for database management. The medical image management system 6 may include a DICOM server that operates based on DICOM specifications. It should be noted that DICOM is an abbreviation for digital imaging and communications in medicine.

The hospital information system 3, the image diagnosis department information management system 4, the modality apparatus 5, and the medical image management system 6 are electrically connected to each other via a network in a communicable manner. The network may be a local area network in the medical facility. A part of the network may be a wide area network. It should be noted that, in FIG. 1, the network is not shown.

The medical image management system 6 receives, via the network, various types of data including the image generated by the modality apparatus 5. The medical image management system 6 stores the received various types of data in a storage medium, such as the large-capacity external storage device. The medical image management system 6 manages the various types of data stored in the storage medium.

The medical image management system 6 performs communication with the hospital information system 3, the image diagnosis department information management system 4, and the like via the network. The medical image management system 6 transmits and receives the various types of data including an examination image to and from the hospital information system 3 or the like. A DICOM protocol can be applied to a storage format of the image data in the medical image management system 6 and communication between the devices via the network.

### Configuration Example of MRI Apparatus

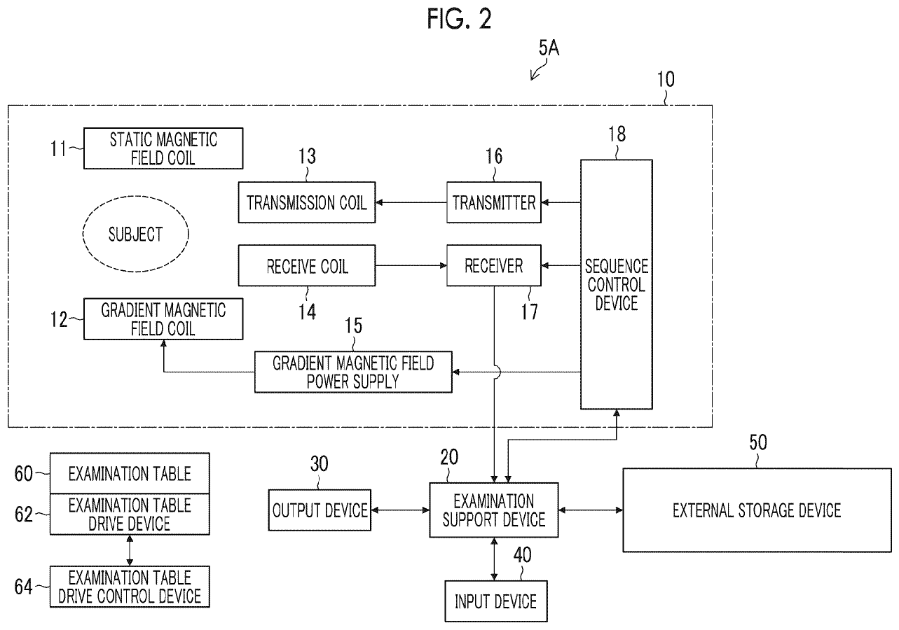

FIG. 2 is an overall configuration diagram of the MRI apparatus applied to the medical information system shown in FIG. 1. An MRI apparatus 5A shown in FIG. 2 is an example of the modality apparatus 5 shown in FIG. 1.

The MRI apparatus 5A shown in FIG. 2 comprises a measurement device 10, an examination support device 20, an output device 30, an input device 40, and an external storage device 50. The measurement device 10 comprises a static magnetic field coil 11, a gradient magnetic field coil 12, and a gradient magnetic field power supply 15. The static magnetic field coil 11 generates a static magnetic field in a space in which the subject is placed. The gradient magnetic field coil 12 provides a magnetic field gradient with respect to the static magnetic field generated by the static magnetic field coil 11. The gradient magnetic field power supply 15 is a drive power supply of the gradient magnetic field coil 12.

The measurement device 10 comprises a transmission coil 13, a receive coil 14, a transmitter 16, and a receiver 17. The transmission coil 13 generates a high-frequency magnetic field in a measurement area of the subject. The transmitter 16 supplies a pulse current, which is an excited current, to the transmission coil 13. The receive coil 14 receives a nuclear magnetic resonance signal generated from the subject. The receiver 17 transmits the nuclear magnetic resonance signal received by using the receive coil 14 to the examination support device 20. It should be noted that the nuclear magnetic resonance signal can be referred to as an echo signal. Any one of a vertical magnetic field method or a horizontal magnetic field method is applied to the MRI apparatus 5A depending on a direction of the static magnetic field to be generated. The static magnetic field coil 11 is adopted in various forms in accordance with the above-described magnetic field method. The gradient magnetic field coil 12 comprises a plurality of coils that generate gradient magnetic fields respectively for three axial directions orthogonal to each other. The plurality of coils provided in the gradient magnetic field coil 12 are each driven by the gradient magnetic field power supply 15. Position information is added to the nuclear magnetic resonance signal generated from the subject due to the application of the gradient magnetic field.

It should be noted that, although FIG. 2 shows an aspect in which the transmission coil 13 and the receive coil 14 are individually provided, there may be an aspect in which one coil having a function of the transmission coil 13 and a function of the receive coil 14 is provided.

The measurement device 10 comprises a sequence control device 18. The sequence control device 18 controls the operations of the gradient magnetic field power supply 15 and the transmitter 16 to control the generation timings of the gradient magnetic field and the high-frequency magnetic field. The sequence control device 18 controls the operation of the receiver 17 to control the reception timing of the nuclear magnetic resonance signal and to execute the measurement. A time chart of the control applied to the sequence control device 18 is referred to as an imaging sequence, is set in advance in accordance with the measurement, and is stored in the storage device or the like provided in the examination support device 20.

As the examination support device 20, a computer comprising one or more processors such as a central processing unit (CPU), one or more memories, a storage device, and the like is applied. The examination support device 20 functions as a control device that controls the operation of each unit of the measurement device 10 via the sequence control device 18. In addition, the examination support device 20 functions as an operation device that executes operation processing on the nuclear magnetic resonance signal received via the receiver 17 and the sequence control device 18 and that acquires an image signal of a predetermined imaging area. The examination support device 20 may be a device that constitutes the MRI apparatus 5A or may be an external device that is independent of the MRI apparatus 5A.

The output device 30, the input device 40, and the external storage device 50 are electrically connected to the examination support device 20 in a data communicable manner. As the output device 30, a display that displays a result of the operation processing executed by using the examination support device 20 can be applied. For example, the output device 30 may display a reconstruction image reconstructed based on the nuclear magnetic resonance signal transmitted from the measurement device 10. It should be noted that the nuclear magnetic resonance signal can be referred to as NMR signal using an abbreviation for nuclear magnetic resonance that is the English notation.

A liquid crystal display, an organic EL display, a projector, and the like can be applied to the output device 30. The output device 30 may be appropriately combined with the liquid crystal display, the organic EL display, the projector, and the like. It should be noted that the organic EL can be referred to as OEL using an abbreviation for organic electro-luminescence. The display in the following description is a display that functions as the output device 30 shown in FIG. 2.

The input device 40 is an interface through which an operator inputs a condition applied to the measurement, a condition applied to the operation processing, parameters, and the like. Examples of the input device 40 include a keyboard and a mouse. The display device that functions as the output device 30 and the input device 40 may be integrally configured by using a touch panel type display. The operator can input the parameters, such as the number of measured echoes, an echo time, and an echo interval, by using the input device 40.

The external storage device 50 stores data used in various types of the operation processing executed by the examination support device 20, data derived as a result of the operation processing, the conditions applied to the operation processing, the parameters applied to the operation processing, a program for executing the operation processing, and the like. The external storage device 50 may realize some of the functions of an internal storage device of the examination support device 20, or the internal storage device of the examination support device 20 may realize some of the functions of the external storage device 50.

The MRI apparatus 5A comprises an examination table 60, an examination table drive device 62, and an examination table drive control device 64. The examination table 60 has a structure on which a subject (subject) is placed, and is configured to move in an up-down direction, a direction of entering an imaging area, and a direction of retreating from the imaging area by using the examination table drive device 62.

The examination table drive device 62 comprises a drive mechanism that is connected to the examination table 60 and a drive source such as a motor that is connected to the drive mechanism. It should be noted that the drive mechanism and the drive source are not shown.

The examination table drive control device 64 controls the operation of the examination table drive device 62 by applying a determined drive condition, and moves the examination table 60 in a determined direction and stops the examination table 60 at a determined position. As the examination table drive control device 64, a computer is applied. The examination table drive control device 64 may comprise an input device, such as a keyboard, and an output device, such as a display.

The examination support device 20 and the examination table drive control device 64 may be configured as a console unit. The console unit functions as various control devices in the MRI apparatus 5A. The console unit is realized by using one or more computers.

### Configuration Example of Examination Support Device According to First Embodiment

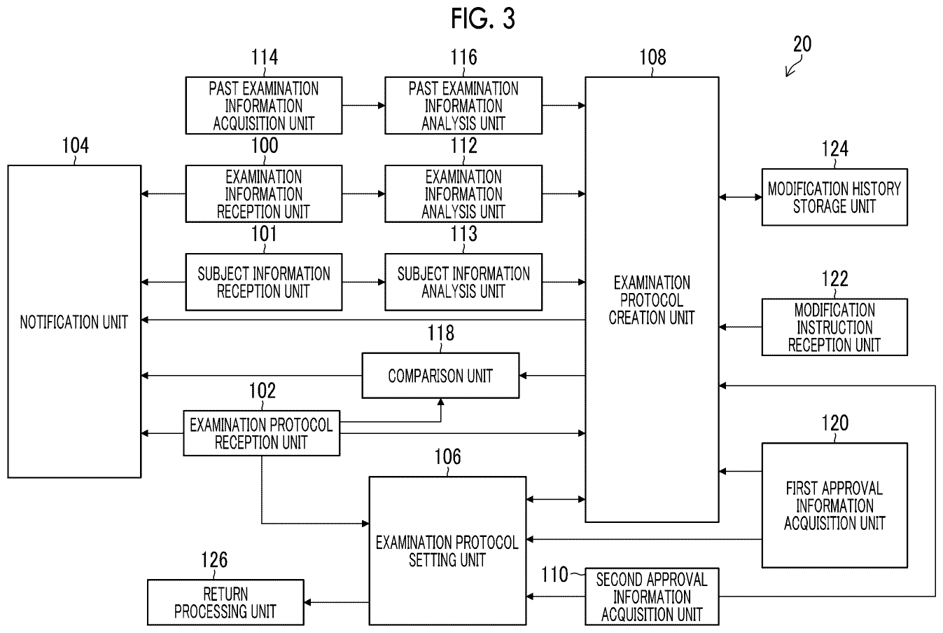

FIG. 3 is a functional block diagram showing an electric configuration of the examination support device according to a first embodiment. The examination support device 20 comprises an examination information reception unit 100. The examination information reception unit 100 receives examination information included in the examination order transmitted from the image diagnosis department information management system 4 shown in FIG. 1. It should be noted that the reception of the examination order is synonymous with the reception of the examination order, the acquisition of the examination order, and the like. The substance of the information in the following description is an electric signal representing the information.

The examination information can include an examination part, an examination type, an examination purpose, and the presence or absence of an examination request to another modality. Examples of the examination information include an example in which the examination part is the brain, the examination type is a non-contrast MRI examination, and the examination purpose is a suspected cerebral infarction.

The examination support device 20 comprises a subject information reception unit 101. The subject information reception unit 101 receives subject information included in the examination order. The subject information includes a physique of the subject, a height of the subject, a necessity of a pre-examination, and a physical feature. Information representing a feature of the physique of the subject, such as tall and thin, may be applied to the physique of the subject.

Examples of the necessity of the pre-examination include information representing whether or not the pre-examination, such as an electrocardiogram examination and a cognitive function examination, is necessary before the MRI examination. Examples of the physical feature include the presence or absence of an in-vivo metal, the presence or absence of an allergy, whether or not breath holding is difficult, and whether or not stillness is difficult. Examples of the in-vivo metal include a metal for fixing a fracture part. It should be noted that the subject according to the embodiment is an example of a subject of the examination, and the subject information according to the embodiment is an example of subject information.

The examination information reception unit 100 and the subject information reception unit 101 function as constituent elements of an examination order reception unit that receives the examination order transmitted from the image diagnosis department information management system 4.

The examination support device 20 comprises an examination protocol reception unit 102. The examination protocol reception unit 102 receives a second examination protocol selected in a case in which the examination order is created in the image diagnosis department information management system 4. For example, the examination protocol reception unit 102 may receive, as the second examination protocol, the examination protocol selected by a doctor in a case in which the examination order is created. It should be noted that the examination protocol can include an imaging protocol applied to the capturing of the medical image. The examination protocol can be simply referred to as a protocol.

The second examination protocol is associated with the examination order including the examination information in advance. That is, one or more second examination protocols are determined depending on the examination part, the examination method, and the examination purpose. A correspondence relationship between the second examination protocol and the examination order is shared between the image diagnosis department information management system 4 and the modality apparatus 5.

Here, one or more sequences are included in the examination protocol. The sequence includes the above-described imaging sequence. For example, in a case in which a plurality of examination protocols having different sequences and a plurality of examination protocols having different combinations of sequences are stored in the modality apparatus 5, individual identification information can be given to and managed for each of the plurality of examination protocols. The identification information for each examination protocol in the modality apparatus 5 may be shared between the image diagnosis department information management system 4 and the modality apparatus 5.

The second examination protocol may be included in the examination order. That is, the examination protocol reception unit 102 may receive the second examination protocol included in the examination order. The examination protocol reception unit 102 can function as a constituent element of the examination order reception unit.

The examination support device 20 comprises a notification unit 104. The notification unit 104 can comprise a notification creation unit that creates the notification, and a notification display controller that generates a display signal representing the notification and transmits the display signal to the display.

The notification unit 104 sends notification of the reception of the examination order. That is, the notification unit 104 sends notification of the reception of the examination information and the reception of the subject information. The notification unit 104 may display text information representing the reception of the examination order on the display as the notification of the reception of the examination order. The notification unit 104 may send notification of a content of the examination information by applying the text information. The notification unit 104 may send notification of a content of the subject information by applying the text information.

The notification unit 104 sends notification of the reception of the second examination protocol. The notification unit 104 may display text information representing the reception of the second examination protocol on the display as the notification of the reception of the second examination protocol. The notification unit 104 may display text information representing a content of the second examination protocol on the display as the notification of the content of the second examination protocol.

The examination support device 20 comprises an examination protocol setting unit 106. The examination protocol setting unit 106 sets the examination protocol applied to the examination, such as setting of the imaging protocol applied to the capturing of the medical image in the modality apparatus 5. For example, the examination protocol setting unit 106 may set, as the examination protocol, the second examination protocol that is received by using the examination protocol reception unit 102 and for which the notification of the reception is sent by using the notification unit 104.

The examination support device 20 comprises an examination protocol creation unit 108. In a case in which the second examination protocol is disapproved as the examination protocol, the examination protocol creation unit 108 creates a first examination protocol to replace the second examination protocol.

The examination order including the examination information and the second examination protocol are associated with each other in advance, but there may be a case in which the second examination protocol needs to be customized depending on the subject. Examples of the case include a case in which the examination order is incorrect and a case in which the examination order in which the necessary examination condition is overlooked is created, and the first examination protocol is automatically created in a case in which the second examination protocol is disapproved.

As the creation of the first examination protocol, the modification of the second examination protocol may be performed. The modification of the second examination protocol includes at least any of the change, the addition, or the deletion of a sequence constituting the second examination protocol. Hereinafter, the term “creation of an examination protocol” can include a concept of modification, change, and the like of the examination protocol.

The examination support device 20 comprises a second approval information acquisition unit 110. The second approval information acquisition unit 110 acquires approval information representing whether or not the second examination protocol for which the notification is sent is approved or disapproved. The approval information can be transmitted by the operator such as a radiologist who has confirmed the correspondence relationship between the examination order and the second examination protocol, via the input device 40 shown in FIG. 2.

In a case in which the approval information representing the approval of the second examination protocol is acquired, the second approval information acquisition unit 110 transmits the approval information to the examination protocol setting unit 106. The examination protocol setting unit 106 sets, as the examination protocol, the second examination protocol based on the approval information.

In a case in which disapproval information representing the disapproval of the second examination protocol is acquired, the second approval information acquisition unit 110 transmits the disapproval information to the examination protocol creation unit 108. The examination protocol creation unit 108 creates the first examination protocol based on the disapproval information. For example, in a case in which the second examination protocol needs to be reviewed, the operator can operate the input device 40 shown in FIG. 2 to transmit the disapproval information of the second examination protocol to the second approval information acquisition unit 110.

The examination support device 20 comprises an examination information analysis unit 112. The examination information analysis unit 112 analyzes the examination information. The examination information analysis unit 112 transmits an analysis result of the examination information to the examination protocol creation unit 108. Examples of the analysis of the examination information include processing of extracting the examination part, the examination method, the examination purpose, and the like.

The examination support device 20 comprises a subject information analysis unit 113. The subject information analysis unit 113 analyzes the subject information. The subject information analysis unit 113 transmits an analysis result of the subject information to the examination protocol creation unit 108. Examples of the analysis of the subject information include processing of extracting the physique, the height, the weight, the presence or absence of the pre-examination, the physical feature, and the like of the subject from the examination order.

The examination support device 20 comprises a past examination information acquisition unit 114 and a past examination information analysis unit 116. The past examination information acquisition unit 114 acquires past examination information regarding a past examination performed in the past on the subject described in the examination order. The past examination information analysis unit 116 analyzes the past examination information. The past examination information analysis unit 116 transmits an analysis result of the past examination information to the examination protocol creation unit 108.

The past examination information may be acquired from the electronic medical record system provided in the hospital information system 3 shown in FIG. 1, or may be acquired from a medical image storage device provided in the medical image management system 6. The past examination information may be acquired from the information stored in the modality apparatus 5.

Examples of the analysis of the past examination information include extraction of the examination protocol applied to the past examination, a comment in the past examination, a past image acquired in the past examination, and the presence or absence of an interpretation request.

The past examination information may include past similar examination information in a past similar examination similar to the past examination performed in the past. Examples of the past similar examination include an examination in which the examination part is different, an examination in which the examination part is the same, and an examination in which the examination condition is different, on the subject described in the examination order.

Examples of the past similar examination information include a creation record of the examination protocol and a modification record of the examination protocol in the similar examination. The past examination information analysis unit 116 may analyze the past similar examination information.

The examination protocol creation unit 108 creates the first examination protocol by using the analysis result of the examination information and the analysis result of the subject information. In a case of the creation of the first examination protocol, the examination protocol creation unit 108 may use the analysis result of the past examination information.

Examples of the creation of the first examination protocol include the addition of a caution before the examination, change in an imaging order in a case of a specific disease, determination of left and right of limbs from the past examination image, determination of positioning of an imaging position from the past examination image, the addition of post-processing after the imaging, the addition of a transmission destination of the examination image based on the presence or absence of the interpretation request, and the addition of a transmission destination of the past examination image based on the presence or absence of the interpretation request.

For example, in a case in which the metal is present in the body of the subject, the addition of the output of a caution message representing that the MRI examination cannot be performed or the addition of the output of a caution message representing that an imaging task is changed is performed.

In a case in which the subject has an allergy to a contrast agent, an output of a message representing that the contrast agent cannot be used is added. In a case of the subject who requires assistance in a bed, a message representing that the subject requires the assistance in the bed is output.

In a case in which the examination purpose is the suspected cerebral infarction, the sequence order is changed and a DWI sequence is executed first. It should be noted that DWI is an abbreviation for diffusion-weighted imaging that is the English notation. That is, specific imaging is first performed depending on the examination part, the examination purpose, and the like.

In a case in which the examination part is not designated in the examination order, the examination part is designated with reference to the past examination image. In a case in which the processing performed in the past examination, such as the three-dimensional analysis, the lesion detection, and the report creation, is not included, the processing performed in the past examination is added. In a case in which the transmission destination of the examination image is not included, as the transmission destination of the examination image, the transmission destination of the past examination image is added.

The notification unit 104 sends notification of the first examination protocol. That is, the notification unit 104 can display, on the display, a GUI for confirming whether or not to approve the first examination protocol. The operator can operate the input device 40 to input the approval or the disapproval of the first examination protocol.

In a case of displaying the GUI for confirming the approval of the first examination protocol, the notification unit 104 may display the second examination protocol on the GUI. It should be noted that GUI is an abbreviation for graphical user interface.

The examination support device 20 comprises a comparison unit 118. The comparison unit 118 compares the second examination protocol with the first examination protocol, to determine the appropriateness of the first examination protocol. The notification unit 104 displays, on the display, a comparison result between the second examination protocol and the first examination protocol.

The examination support device 20 comprises a first approval information acquisition unit 120. The first approval information acquisition unit 120 acquires information representing the approval or the disapproval of the first examination protocol. In a case in which the first approval information acquisition unit 120 acquires the approval information representing the approval of the first examination protocol, the examination protocol setting unit 106 sets the first examination protocol as the examination protocol.

In a case in which the first approval information acquisition unit 120 acquires the disapproval information representing the disapproval of the first examination protocol, the examination protocol creation unit 108 can modify the first examination protocol. In a case in which the first approval information acquisition unit 120 acquires the disapproval information representing the disapproval of the first examination protocol, the examination protocol setting unit 106 can return the second examination protocol to the image diagnosis department information management system 4.

The examination support device 20 comprises a modification instruction reception unit 122. The modification instruction reception unit 122 receives a modification instruction of the first examination protocol. For example, the modification instruction reception unit 122 acquires the modification instruction of the first examination protocol input from the operator by operating the input device 40.

The modification instruction reception unit 122 transmits the modification instruction of the first examination protocol to the examination protocol creation unit 108. The examination protocol creation unit 108 modifies the first examination protocol based on the received modification instruction. The notification unit 104 may display, on the display, a GUI for confirming whether or not to approve the examination protocol in which the first examination protocol is modified.

The examination support device 20 comprises a modification history storage unit 124. The modification history storage unit 124 stores a modification history of the examination protocol performed by the examination protocol creation unit 108.

The modification history storage unit 124 can store, as the modification history, the operator who has actually approved or disapproved the examination protocol, a modification portion of the examination protocol, a modification portion of the captured medical image, a comment in a case of the modification, the number of modifications, and the like.

The examination support device 20 comprises a return processing unit 126. The return processing unit 126 returns the second examination protocol to the image diagnosis department information management system 4 in a case in which the first approval information acquisition unit 120 acquires the disapproval information representing the disapproval of the first examination protocol and a determined return condition is satisfied. Examples of the return condition include a case in which the examination protocol after the modification is not approved even though a determined number of modifications are performed.

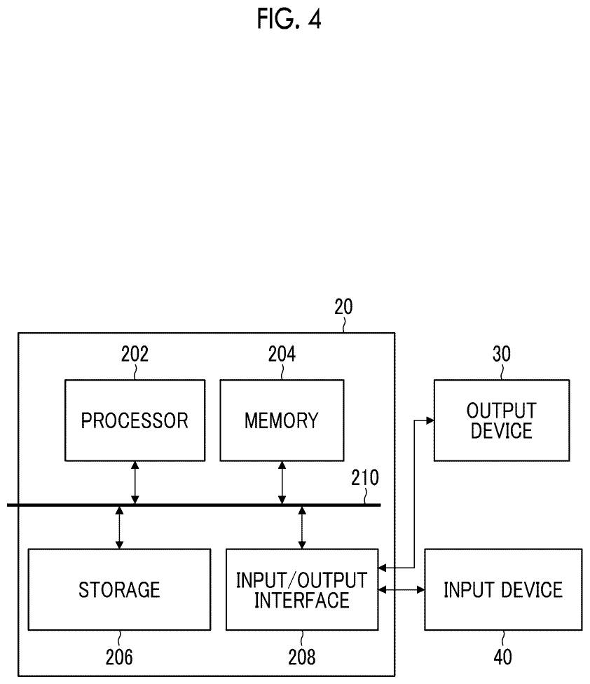

FIG. 4 is a block diagram showing a hardware configuration of the electric configuration of the examination support device shown in FIG. 3. The computer applied to the examination support device 20 may be a personal computer or a workstation. The computer may be a virtual machine. The examination support device 20 may be configured by using a plurality of computers.

The examination support device 20 comprises a processor 202, a memory 204 as a main storage device, a storage 206 as an auxiliary storage device, an input/output interface 208, and a bus 210. The processor 202 includes a CPU. The processor 202 may include a GPU. It should be noted that the CPU is an abbreviation for central processing unit. GPU is an abbreviation for graphics processing unit.

The processor 202 is connected to the memory 204, the storage 206, and the input/output interface 208 via the bus 210. The input device 40 and the output device 30 are connected to the examination support device 20 via the input/output interface 208.

The processor 202 executes a program including one or more commands stored in the memory 204 to realize various functions. The input/output interface 208 includes a communication interface connectable to the network, a connection interface connectable to an external device, and the like. As the connection interface connectable to the external device, for example, a universal serial bus or HDMI can be applied. It should be noted that the universal serial bus can be referred to as USB that is an abbreviation for universal serial bus. HDMI is an abbreviation for high-definition multimedia interface. USB and HDMI are registered trademarks.

The processor 202 communicates with various external devices of the MRI apparatus 5A via the input/output interface 208, to transmit and receive necessary information. It should be noted that various external devices are not shown.

The output device 30 can include a display that displays various types of information in addition to the medical image captured by using the modality apparatus 5. The output device 30 is used as a part of a GUI in a case of receiving the input from the input device 40. It should be noted that the number of output devices 30 is not limited to one, and a multi-display form comprising a plurality of displays can also be adopted.

### Procedure of Examination Support Method According to First Embodiment

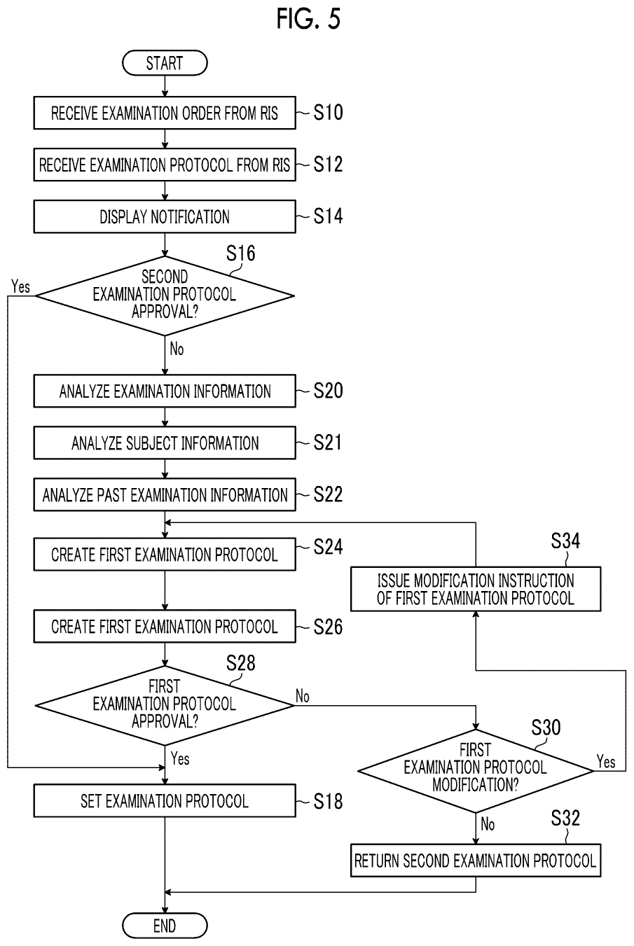

FIG. 5 is a flowchart showing a procedure of an examination support method according to the first embodiment. In an examination order reception step S10, the examination information reception unit 100 shown in FIG. 3 receives the examination information included in the examination order transmitted from the image diagnosis department information management system 4. In the examination order reception step S10, the subject information reception unit 101 receives the subject information included in the examination order transmitted from the image diagnosis department information management system 4.

An examination information reception step of receiving the examination information and a subject information reception step of receiving the subject information are included in the examination order reception step S10. After the examination order reception step S10, the procedure of the examination support method proceeds to an examination protocol reception step S12.

In the examination protocol reception step S12, the examination protocol reception unit 102 receives the examination protocol transmitted from the image diagnosis department information management system 4. The order of the examination order reception step S10 and the examination protocol reception step S12 may be changed, or the examination order reception step S10 and the examination protocol reception step S12 may be executed in parallel. After the examination protocol reception step S12, the procedure of the examination support method proceeds to a notification display step S14.

In the notification display step S14, the notification unit 104 sends the notification of the reception of the examination order and sends the notification of the reception of the examination protocol. In the notification display step S14, an examination order notification step of sending notification of the reception of the examination order and an examination protocol notification step may be executed as separate steps. After the notification display step S14, the procedure of the examination support method proceeds to a second examination protocol approval determination step S16.

The operator who has confirmed the reception of the examination order and the second examination protocol for which the notification is sent in the notification display step S14 can confirm whether or not the second examination protocol is appropriate for the examination order.

In the second examination protocol approval determination step S16, the second approval information acquisition unit 110 determines whether to acquire the approval information representing the approval of the second examination protocol or to acquire the disapproval information representing the disapproval of the second examination protocol.

In a case in which it is determined in the second examination protocol approval determination step S16 that the approval information representing the approval of the second examination protocol is acquired, a Yes determination is made, and the procedure of the examination support method proceeds to an examination protocol setting step S18.

In the examination protocol setting step S18, the examination protocol setting unit 106 sets the second examination protocol as the examination protocol. In a case in which the examination protocol applied to the examination is set in the examination protocol setting step S18, determined end processing is executed, and then the procedure of the examination support method ends.

On the other hand, in a case in which it is determined in the second examination protocol approval determination step S16 that the disapproval information representing the disapproval of the second examination protocol is acquired, a No determination is made, and the procedure of the examination support method proceeds to an examination information analysis step S20.

That is, the operator who has confirmed whether or not the second examination protocol is appropriate for the examination order may set the received second examination protocol as the examination protocol applied to the examination, or may modify the second examination protocol. In a case in which the modification of the second examination protocol is selected, the following procedure is applied, and the first examination protocol in which the second examination protocol is modified is automatically generated.

In the examination information analysis step S20, the examination information analysis unit 112 analyzes the examination information. The analysis result of the examination information in the examination information analysis step S20 is transmitted to the examination protocol creation unit 108. After the examination information analysis step S20, the procedure of the examination support method proceeds to a subject information analysis step S21.

In the subject information analysis step S21, the subject information analysis unit 113 analyzes the subject information. The analysis result of the subject information in the subject information analysis step S21 is transmitted to the examination protocol creation unit 108. After the subject information analysis step S21, the procedure of the examination support method proceeds to a past examination information analysis step S22. The order of the examination information analysis step S20 and the subject information analysis step S21 may be changed, or the examination information analysis step S20 and the subject information analysis step S21 may be executed in parallel.

In the past examination information analysis step S22, the past examination information analysis unit 116 acquires the past examination information to analyze the past examination information. The analysis result of the past examination information in the past examination information analysis step S22 is transmitted to the examination protocol creation unit 108. A past examination information acquisition step of acquiring the past examination information can be included in the past examination information analysis step S22. After the past examination information analysis step S22, the procedure of the examination support method proceeds to an examination protocol creation step S24.

In the examination protocol creation step S24, based on the analysis result of the examination information and the analysis result of the subject information, the examination protocol creation unit 108 creates the first examination protocol in which at least a part of the second examination protocol is changed. The analysis result of the past examination information may be used in a case of the creation of the first examination protocol. After the examination protocol creation step S24, the procedure of the examination support method proceeds to a first examination protocol notification step S26.

In the first examination protocol notification step S26, the notification unit 104 displays the notification of the first examination protocol created in the examination protocol creation step S24. That is, in the first examination protocol notification step S26, the first examination protocol is displayed on the display.

In the first examination protocol notification step S26, the first examination protocol and the second examination protocol may be displayed. In a case in which the first examination protocol is displayed, a comparison step of comparing the second examination protocol with the first examination protocol may be executed to display the comparison result between the second examination protocol and the first examination protocol. After the first examination protocol notification step S26, the procedure of the examination support method proceeds to a first examination protocol approval determination step S28.

That is, the operator who has confirmed the first examination protocol displayed on the display may set the first examination protocol as the examination protocol applied to the examination, or may further modify the first examination protocol.

In the first examination protocol approval determination step S28, the first approval information acquisition unit 120 determines whether to acquire the approval information representing the approval of the first examination protocol or to acquire the disapproval information representing the disapproval of the first examination protocol.

In a case in which it is determined in the first examination protocol approval determination step S28 that the approval information representing the approval of the first examination protocol is acquired, a Yes determination is made, and the procedure of the examination support method proceeds to an examination protocol setting step S18, and the first examination protocol is set as the examination protocol applied to the examination in the examination protocol setting step S18.

On the other hand, in a case in which it is determined in the first examination protocol approval determination step S28 that the disapproval information representing the disapproval of the second examination protocol is acquired, a No determination is made, and the procedure of the examination support method proceeds to a modification determination step S30.

That is, in the first examination protocol approval determination step S28, the received second examination protocol and the created first examination protocol are compared with each other, and the operator determines the appropriateness of the created first examination protocol. The examination support device 20 displays a GUI for confirming the appropriateness of the first examination protocol on the display.

The examination support device 20 displays the first examination protocol and the second examination protocol in an arranged manner on the GUI. In a case in which the sequence of the first examination protocol and the sequence of the second examination protocol are different from each other, the examination support device 20 displays the information included in the examination order, the change reason of the sequence, and the like on the GUI. The change portion and the change reason may be changed in a display aspect with other information. Examples of the change in the display aspect include a change in color and the use of emphasized text.

For example, in a case in which the in-vivo metal is overlooked in the examination order and is substituted with the imaging sequence in which a metal artifact is relatively reduced in the first examination protocol, the examination support device 20 may display the text information representing the in-vivo metal in the examination information on the GUI. As a result, the operator of the modality apparatus 5 can confirm the change reason of the sequence.

In the modification determination step S30, the examination protocol setting unit 106 determines whether or not to perform the modification of the first examination protocol or to return the second examination protocol to the image diagnosis department information management system 4. In a case in which it is determined in the modification determination step S30 that the second examination protocol is returned to the image diagnosis department information management system 4, a No determination is made, and the procedure proceeds to a second examination protocol return step S32.

In the second examination protocol return step S32, the second examination protocol is returned to the image diagnosis department information management system 4. In a case in which the second examination protocol is returned, the examination order may be returned. In a case in which the second examination protocol is returned in the second examination protocol return step S32, determined end processing is executed, and then the procedure of the examination support method ends.

On the other hand, in a case in which it is determined in the modification determination step S30 that the modification of the first examination protocol is executed, a Yes determination is made, and the procedure proceeds to a modification instruction reception step S34. In the modification instruction reception step S34, the modification instruction reception unit 122 receives the modification instruction of the first examination protocol. The modification instruction of the first examination protocol is transmitted to the examination protocol creation unit 108. After the modification instruction reception step S34, the procedure proceeds to the examination protocol creation step S24, and the modification of the first examination protocol is executed until the modified first examination protocol is approved or the second examination protocol is returned.

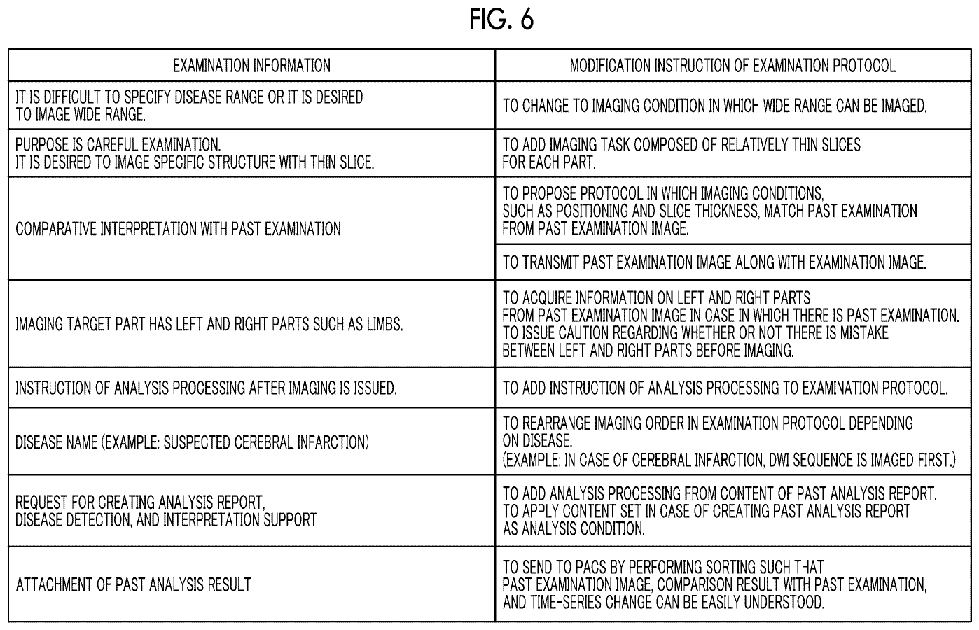

In a case in which the first examination protocol is modified in the examination protocol creation step S24, a modification history storage step of storing the modification history of the first examination protocol may be executed, or a modification history notification step of sending notification of the modification history of the first examination protocol may be executed. It should be noted that the examination support method of which the procedure is shown in FIG. 6 is an example of an operation method of an examination support device.

### Specific Example of Examination Protocol Creation

FIG. 6 is a table showing a correspondence relationship between the examination information and a change content of the examination protocol. In a case in which the examination information corresponding to the left column of the table shown in FIG. 6 is obtained in the examination information analysis step S20 shown in FIG. 5, the modification instruction corresponding to the change content of the examination protocol in the right column of the table of FIG. 6 is received in the modification instruction reception step S34 shown in FIG. 5.

For example, in a case in which it is difficult to specify a disease range or a wide range is imaged, the imaging conditions set in the examination protocol are changed to the imaging conditions in which a relatively wide range can be imaged.

In a case in which the purpose is careful examination of a specific structure, for example, in a case in which it is desired to image a specific structure in the examination part by applying a relatively thin slice, an imaging task composed of the relatively thin slices is added for each part or for each structure in the part.

In a case in which an instruction of the comparative interpretation with the past examination is included in the second examination protocol, a first examination protocol in which the imaging conditions, such as the positioning of the imaging position and a slice thickness from the past examination image, match the imaging conditions of the past examination image is proposed. In addition, the past examination image is transmitted along with the transmission of the examination image.

In a case in which the imaging target part has left and right parts, such as limbs, a caution regarding whether or not there is a mistake between the left and right parts before the imaging is issued. In a case in which the left and right parts are not designated, the information on the left and right parts is acquired from the past examination image in a case in which the past examination image is present.

In a case in which an instruction of the post-processing after the imaging, such as the analysis processing of the examination image, is issued, the post-processing after the imaging is added. In a case in which a disease name is included, the sequences are rearranged in accordance with the disease name. It should be noted that the disease name used herein is synonymous with the examination purpose. For example, in a case in which the disease name is the suspected cerebral infarction, the sequence order is changed, and the DWI sequence is first performed.

In a case in which instruction of a request for creating an analysis report, the lesion detection, the interpretation support, or the like is issued, analysis processing performed in the past examination is added with reference to the content of the past analysis report. The analysis condition set in the past examination is applied as the analysis condition of this time.

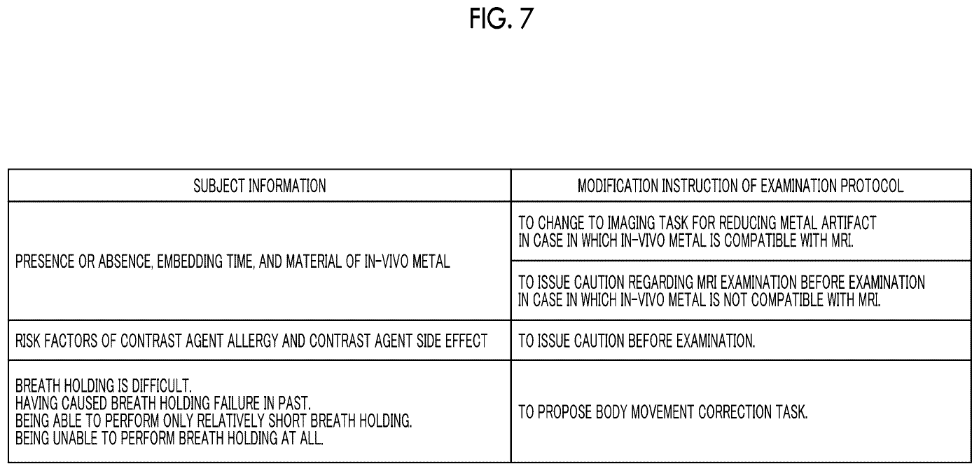

FIG. 7 is a table showing a correspondence relationship between the subject information and the change content of the examination protocol. In a case in which the subject information corresponding to the left column of the table shown in FIG. 7 is obtained in the subject information analysis step S21 shown in FIG. 5, the modification instruction corresponding to the change content of the examination protocol in the right column of the table of FIG. 7 is received in the modification instruction reception step S34 shown in FIG. 5.

In a case in which the presence or absence of the in-vivo metal of the subject, the embedding time of the in-vivo metal, and the material of the in-vivo metal are included in the subject information, in an MRI examination compatible with the in-vivo metal, the change to the imaging task for reducing the metal artifact is performed. In addition, in the MRI examination that is not compatible with the in-vivo metal, a caution representing that the MRI examination is not compatible with the in-vivo metal before the examination is issued. The caution that the MRI examination is not compatible with the in-vivo metal can be understood as the caution representing that the MRI examination cannot be performed.

In a case in which risk factors of the contrast agent allergy and the side effects of the contrast agent are included in the subject information, a caution regarding the contrast agent allergy and the like is issued before the examination. A body movement correction task is proposed in a case in which the subject information includes that the breath holding is difficult, such as having caused the breath holding failure in the past, being able to perform only a relatively short breath holding, and being unable to perform the breath holding at all.

It should be noted that the modification instruction of the examination protocol corresponding to the examination information shown in FIG. 6 and the modification instruction corresponding to the subject information shown in FIG. 7 are merely examples, and may be appropriately added, deleted, and modified.

### Configuration Example of User Interface

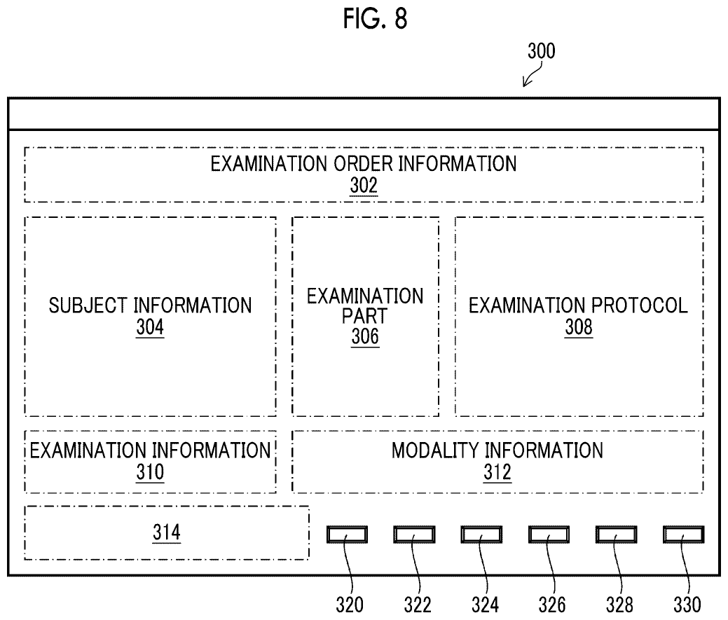

FIG. 8 is a schematic diagram showing a configuration example of a user interface. A screen 300 shown in FIG. 8 is displayed on the display that functions as the output device 30 shown in FIG. 1. That is, the notification unit 104 shown in FIG. 3 transmits the display signal representing the screen 300 to the display.

The screen 300 includes an examination order information area 302, a subject information area 304, an examination part area 306, an examination protocol area 308, an examination information area 310, a modality information area 312, and a text input area 314.

Various types of information on the examination order, such as an examination ID and a creator of the examination order, are displayed in the examination order information area 302. It should be noted that ID is an abbreviation for identification. Various types of information on the subject, such as a name of the subject, a subject ID, a height, and a weight, are displayed in the subject information area 304. In addition, the subject information area 304 displays the subject information used for creating the examination protocol such as the presence or absence of the in-vivo metal shown in FIG. 7.

Information on an examination target part is displayed in the examination part area 306. As the examination target part, an anatomical structure such as an organ and a partial structure included in the anatomical structure may be displayed. The examination part area 306 may display information representing the designation of the left and right parts as the examination target part.

The examination protocol area 308 displays the second examination protocol associated with the examination order. In the examination protocol area 308, the identification information for identifying the type of the second examination protocol may be displayed as the examination protocol.

The examination information area 310 displays the examination information used for creating the examination protocol, such as difficulty in specifying the disease range shown in FIG. 6. The modality information area 312 displays the modality information regarding the modality apparatus 5, such as the setting of the modality apparatus 5. The text input area 314 displays the text information input by the operator using the input device 40 shown in FIG. 2.

The screen 300 displays a first button 320, a second button 322, a third button 324, a fourth button 326, a fifth button 328, and a sixth button 330. Six buttons are shown in FIG. 8, but the number of buttons need only be one or more.

Various types of information input to the modality apparatus 5 are assigned to the first button 320 and the like. For example, the first button 320 may be assigned with the information input representing the approval of the operator on the examination protocol displayed in the examination protocol area 308.

Similarly, the second button 322 may be assigned with the information input representing the modification on the examination protocol displayed in the examination protocol area 308. The third button 324 may be assigned with the information representing the return of the second examination protocol. That is, the first button 320 may function as an approval button, and the second button 322 may function as a modification instruction button. Further, the third button 324 may function as a return button.

For example, in the notification display step S14 shown in FIG. 5, the second examination protocol is displayed in the examination protocol area 308 of the screen 300 shown in FIG. 8. In a case of approving the second examination protocol, the operator can click the first button 320 to transmit the information representing the approval of the second examination protocol to the examination protocol setting unit 106 shown in FIG. 3.

On the other hand, in a case of modifying the second examination protocol, the operator clicks the second button 322 to transmit the disapproval information of the second examination protocol, which is the information on the modification instruction of the second examination protocol, to the examination protocol setting unit 106.

In addition, in the first examination protocol notification step S26, the first examination protocol is displayed in the examination protocol area 308 of the screen 300. In a case of approving the first examination protocol, the operator clicks the first button 320 to transmit information representing the approval of the first examination protocol to the examination protocol setting unit 106 shown in FIG. 3.

On the other hand, in a case of modifying the first examination protocol, the operator clicks the second button 322 to transmit information representing the modification of the first examination protocol to the examination protocol creation unit 108. Further, in a case in which the operator does not modify the first examination protocol and returns of the second examination protocol, the operator clicks the third button 324 to transmit the information representing the return of the second examination protocol to the examination protocol setting unit 106. The examination protocol setting unit 106 transmits a signal representing the return of the second examination protocol to the return processing unit 126.

The comparison result between the second examination protocol and the first examination protocol may be displayed on the screen 300. The examination protocol area 308 may be applied to the display the comparison result between the second examination protocol and the first examination protocol.

It should be noted that the functions of the first button 320, the second button 322, and the third button 324 are merely examples, and the functions of the first button 320 or the like can be set as appropriate. Examples of the functions that can be assigned to the first button 320 and the like include a function of advancing the screen, a function of returning the screen, a function of fixing the input such as text input, and a function of canceling the input. It should be noted that the screen 300 shown in FIG. 8 is an example of an authentication screen for the operator to input whether or not to approve the first examination protocol.

### Actions and Effects of First Embodiment

The examination support device 20 and the examination support method according to the first embodiment can obtain the actions and effects described below.

[1]

The examination support device 20 receives the examination order including the subject information and the examination information, receives the second examination protocol associated with the examination order, and sends notification of the reception of the examination order and the reception of the second examination protocol. In a case in which the disapproval information of the second examination protocol is acquired, the second examination protocol is modified based on the analysis result of the subject information and the analysis result of the examination information to create the first examination protocol. The modality apparatus 5 performs the examination on the subject by applying the first examination protocol. As a result, the operator is prevented from performing the modification of the second examination protocol associated with the examination order, and the load on the operator is reduced.

[2]

The examination support device 20 acquires the past examination information to analyze the past examination information. The examination support device 20 modifies the second examination protocol to create the first examination protocol with reference to the analysis result of the past examination information. As a result, the information necessary for the creation of the first examination protocol can be acquired from the analysis result of the past examination information.

[3]

The examination support device 20 displays the first examination protocol created based on the second examination protocol on the display that functions as the output device 30. As a result, the operator can confirm the content of the first examination protocol.

That is, it is preferable to perform the imaging determined by the operator as being appropriate in the modality apparatus 5, but automatic change of the instruction transmitted from the image diagnosis department information management system 4 is not preferable. Therefore, a mechanism is provided in which the first examination protocol created based on the second examination protocol is displayed, and the operator can confirm the modification content of the second examination protocol. In addition, the second examination protocol can be returned to the image diagnosis department information management system 4 as necessary.

[4]

On the screen 300 on which the first examination protocol is displayed, the first button 320 for outputting the signal representing the approval of the first examination protocol is displayed. As a result, the operator can operate the first button 320 to input the signal representing the approval of the first examination protocol.

[5]

The first examination protocol is created by performing at least any one of the substitution, the order change, or the addition of the sequence in the second examination protocol. As a result, the examination protocol is changed in units of the sequence, and the load on the operator is reduced in the change of the examination protocol.

[6]

The examination support device 20 receives the modification of the first examination protocol. As a result, the first examination protocol appropriate for the examination can be modified, and the load on the operator in the modification of the examination protocol can be reduced.

[7]

The examination support device 20 sends notification of the comparison result between the first examination protocol created based on the second examination protocol, and the second examination protocol. As a result, the operator can determine the appropriateness of the first examination protocol.

[8]

The examination support device 20 returns the second examination protocol associated with the examination order to the image diagnosis department information management system 4. As a result, the image diagnosis department information management system 4 can be notified that there is a problem in the association between the examination order and the second examination protocol.

### Configuration Example of Examination Support Device According to Second Embodiment

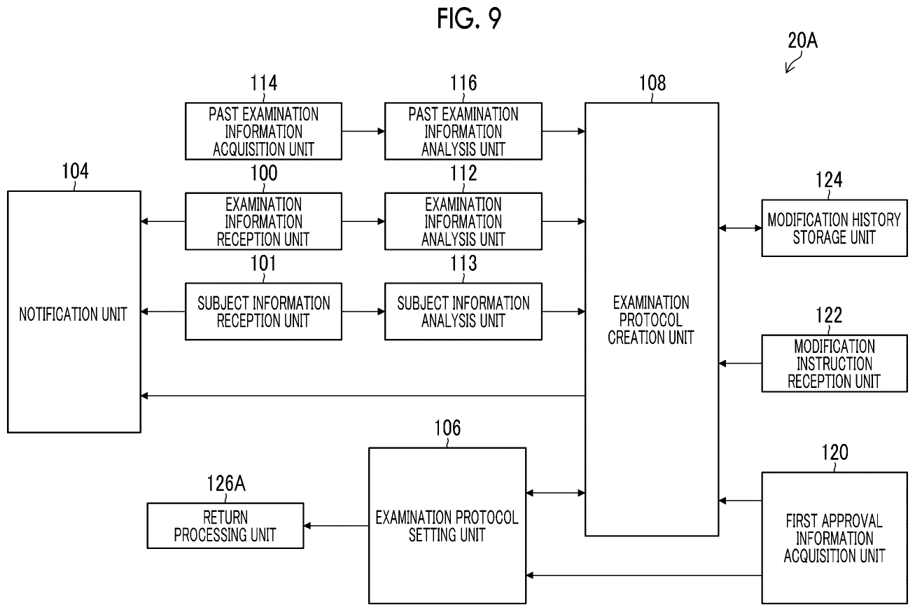

FIG. 9 is a functional block diagram showing an electric configuration of an examination support device according to a second embodiment. An examination support device 20A receive the transmission of the examination order from the image diagnosis department information management system 4, but does not receive the transmission of the second examination protocol. The examination support device 20A creates the first examination protocol based on the examination information and the subject information, and with reference to the past examination information as necessary.

The examination support device 20A does not include the examination protocol reception unit 102, the second approval information acquisition unit 110, and the comparison unit 118 with respect to the examination support device 20 shown in FIG. 3. In addition, in a case in which the first examination protocol is not approved and the modification is not performed, a return processing unit 126A of the examination support device 20A returns the examination order to the image diagnosis department information management system 4.

### Procedure of Examination Support Method According to Second Embodiment

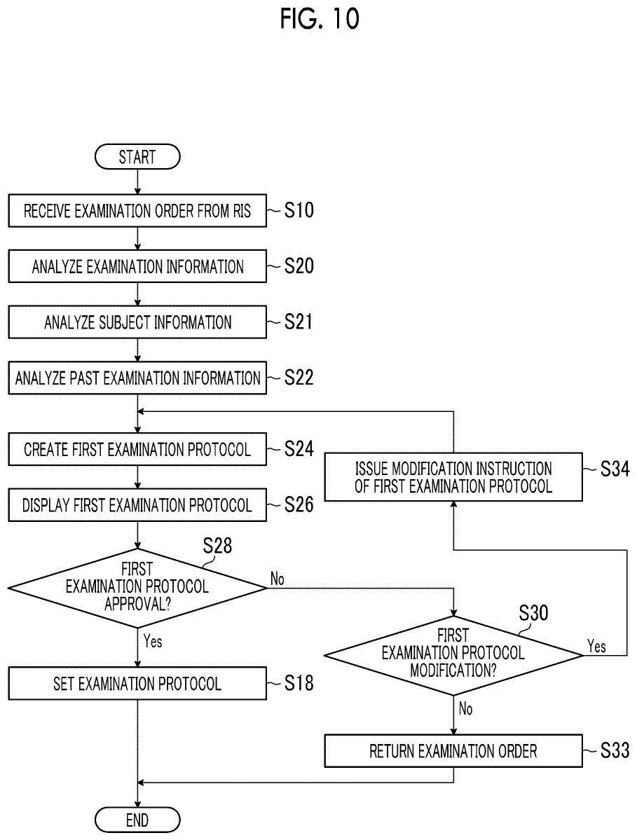

FIG. 10 is a flowchart showing a procedure of an examination support method according to the second embodiment. In the flowchart shown in FIG. 10, the examination protocol reception step S12, the notification display step S14, and the second examination protocol approval determination step S16 shown in FIG. 5 are not included. In addition, instead of the second examination protocol return step S32 of returning the second examination protocol shown in FIG. 5, an examination order return step S33 of returning the examination order is included. It should be noted that the examination support method of which the procedure is shown in FIG. 10 is an example of an operation method of an examination support device.

### Actions and Effects of Second Embodiment

In the examination support device and the examination support method according to the second embodiment, the first examination protocol is automatically created based on the analysis result of the examination order even in a case in which the examination protocol is not transmitted in a case in which the examination order is transmitted. As a result, the operator does not need to analyze the examination order and create the examination protocol based on the analysis result, and the load on the operator is reduced.

### Hardware Configuration of Each Processing Unit

The hardware structure of the processing units that executes various types of processing such as the examination information reception unit 100, the subject information reception unit 101, and the examination protocol reception unit 102 in the examination support device 20 shown in FIG. 3 and the examination support device 20A shown in FIG. 9 is, for example, various processors as follows.

The various processors include, for example, a CPU which is a general-purpose processor executing a program to function as various processing units, a GPU, a programmable logic device (PLD), such as a field programmable gate array (FPGA), which is a processor whose circuit configuration can be changed after manufacture, and a dedicated electric circuit, such as an application specific integrated circuit (ASIC), which is a processor having a dedicated circuit configuration designed to execute specific processing.

One processing unit may be configured by one of the various processors or by two or more processors of the same type or different types. For example, one processing unit may be configured by a plurality of FPGAs, a combination of a CPU and an FPGA, or a combination of a CPU and a GPU. Further, a plurality of processing units may be configured by one processor. A first example of the configuration in which a plurality of processing units are configured by one processor is a form in which one processor is configured by a combination of one or more CPUs and software and this processor functions as a plurality of processing units, as represented by a computer such as a client or a server. A second example of the configuration is a form in which a processor that implements the functions of the entire system including a plurality of processing units using one integrated circuit (IC) chip is used, as represented by a system on chip (SoC). In this way, various processing units are configured by using one or more of the various processors as a hardware structure.

Further, the hardware structure of the various processors is an electric circuit (circuitry) obtained by combining circuit elements such as semiconductor elements.

### Program for Operating Computer

A program causing a computer to realize some or all of the processing functions of the examination support device 20 and the like according to the embodiment can be recorded in a computer-readable medium which is an optical disk, a magnetic disk, a semiconductor memory, or other tangible non-transitory information storage medium, and the program can be provided via the information storage medium.

Instead of the aspect of providing the program by storing the program in the tangible non-transitory computer-readable medium, it is also possible to provide the program as a program signal by using an electric communication line such as the Internet.

Further, some or all of the processing functions in the examination support device 20 or the like may be realized by cloud computing, or may be provided as software as a service (SaaS).

### Example of Application to Medical Image Diagnostic Examination System

The measurement device 10 and the examination support device 20 shown in FIG. 2 may be electrically connected to each other via any network in a data communicable manner to constitute a medical image diagnostic examination system. The data communication between the measurement device 10 and the examination support device 20 may be wired communication or wireless communication. In addition, a computer that functions as the processing unit that creates the first examination protocol shown in FIG. 3 and a computer that functions as the control device of the measurement device 10 may be provided independently of the examination support device 20, and the computer that functions as the processing unit and the computer that functions as the control device of the measurement device 10 may be electrically connected to each other via any network in a data communicable manner.

The present disclosure is not limited to the above-described embodiments, and various modifications can be made without departing from the gist of the technical idea of the present disclosure.

### EXPLANATION OF REFERENCES

1: medical information system

3: hospital information system

4: image diagnosis department information management system

5: modality apparatus

5A: MRI apparatus

6: medical image management system

20: examination support device

30: output device

40: input device

100: examination information reception unit

101: subject information reception unit

102: examination protocol reception unit

104: notification unit

106: examination protocol setting unit

108: examination protocol creation unit

112: examination information analysis unit

113: subject information analysis unit

120: first approval information acquisition unit

What is claimed is:

1. An examination support device comprising:
one or more processors; and
one or more memories that store a program including one or more commands executed by the one or more processors,
wherein the one or more processors execute the program stored in the one or more memories to:
acquire an examination order for a subject of a medical image diagnostic examination;
acquire subject information regarding the subject;
acquire examination information regarding the medical image diagnostic examination performed on the subject; and
create a first examination protocol applied to the medical image diagnostic examination performed based on the examination order, based on the subject information and the examination information.

2. The examination support device according to claim 1,
wherein the one or more processors
acquire a second examination protocol associated with the examination order, and
create the first examination protocol by modifying the second examination protocol based on the examination information and the subject information.

3. The examination support device according to claim 2,
wherein the one or more processors display, on a display device, a comparison result obtained by comparing the first examination protocol with the second examination protocol.

4. The examination support device according to claim 2,
wherein the one or more processors perform at least any one of substitution of a sequence, order change of a sequence, addition of a sequence, or deletion of a sequence in the second examination protocol in a case of creating the first examination protocol.

5. The examination support device according to claim 1,
wherein the one or more processors
acquire past examination information regarding a past medical image diagnostic examination performed in past on the subject, and
create the first examination protocol based on the subject information, the examination information, and the past examination information.

6. The examination support device according to claim 1,
wherein the one or more processors display, on a display device, an authentication screen for an operator to input whether or not to approve the first examination protocol.

7. The examination support device according to claim 1,
wherein the one or more processors receive modification of the created first examination protocol.

8. The examination support device according to claim 7,
wherein the one or more processors store a modification history of the first examination protocol.

9. An operation method of an examination support device, the operation method comprising:
acquiring an examination order for a subject of a medical image diagnostic examination;
acquiring subject information regarding the subject;
acquiring examination information regarding the medical image diagnostic examination performed on the subject; and
creating a first examination protocol applied to the medical image diagnostic examination performed based on the examination order, based on the subject information and the examination information.

10. A non-transitory, computer-readable tangible recording medium which records thereon a program for causing, when read by a computer, the computer to realize:
a function of acquiring an examination order for a subject of a medical image diagnostic examination;
a function of acquiring subject information regarding the subject;
a function of acquiring examination information regarding the medical image diagnostic examination performed on the subject; and
a function of creating a first examination protocol applied to the medical image diagnostic examination performed based on the examination order, based on the subject information and the examination information.

11. A medical image capturing apparatus comprising:
an examination support device that supports capturing of a medical image,
wherein the examination support device includes:
one or more processors; and
one or more memories that store a program including one or more commands executed by the one or more processors,
the one or more processors execute the program stored in the one or more memories to:
acquire an examination order for a subject of a medical image diagnostic examination;
acquire subject information regarding the subject;
acquire examination information regarding the medical image diagnostic examination performed on the subject; and
create a first examination protocol applied to the medical image diagnostic examination performed based on the examination order, based on the subject information and the examination information.
